(19) United States
(12) Patent Application Publication
(10) Pub. No.: US 2010/0040761 A1
(43) Pub. Date: Feb. 18, 2010

(54) Novel Thraustochytrium SP, KJS-I, Bacillus Polyfermenticus KJS-2 and Feed Additive For Fish Including them

(75) Inventors: Jae-Seon Kang, Busan (KR); Chun-Gyu Kim, Gimhae (KR); Kang-Min Kim, Busan (KR); Jin-Young Lee, Busan (KR); Dong-Hee Kim, Gimhae (KR); Jae-Young Baek, Busan (KR); Ju-Han Lee, Daejeon (KR); Dong-Kyung Lee, Busan (KR); Tae-Sung Jung, Busan (KR); You-Soo Park, Busan (KR); Dong-Hun Kim, Busan (KR)

Correspondence Address: Revolution IP, PLLC, 5234 Capon Hill Place, Burke, VA, 22015, US

(73) Assignees: Jae-Seon Kang, Busan (KE); YOO YOUNG PHARM. CO. LTD,, Jincheon (KR)

(21) Appl. No.: 12/443,691

(22) PCT Filed: Dec. 8, 2006
(86) PCT No.: PCT/KR2006/005339

§ 371 Date: Mar. 30, 2009

(30) Foreign Application Priority Data
Oct. 2, 2006 (KR) 10-2006-0096935

(51) Int. Cl. C12N 1/20; C12N 1/00; A23L 1/30

(52) U.S. Cl. 426/648; 435/243; 435/252.5

(57) ABSTRACT

Disclosed herein are novel microbes Thraustochytrium sp. KJS-1 and Bacillus polyfermenticus KJS-2. Also provided is an aquaculture feed additive for use in fisheries, which comprises freeze dried Thraustochytrium sp. KJS-1, freeze dried Bacillus polyfermenticus KJS- 2, and freeze dried Bacillus licheniformis. In addition to providing DHA, an essential nutrient for fish, the aquaculture feed additive shows antibacterial and antifungal activity, thereby preventing fish from dying from bacterial and fungal diseases.

### TECHNICAL FIELD

The present invention relates to novel microbes Thraustochytrium sp. KJS-1 and Bacillus polyfermenticus KJS-2, and an aquaculture feed additive for fisheries, comprising the novel microbes plus Bacillus licheniformis.

### BACKGROUND ART

Aquaculture feed additives for fisheries have been used for various purposes, including the provision of bacterial resistance to the fish and the sanitization of the fisheries. Also, DHA (docosahexanoic acid)-administered plankton was reportedly used to provide DHA for fish. Artemia or rotifers have been cultured as aquaculture feed for fish, especially fry.

When fish are deficient in DHA, an essential nutrient therefor, they are known to suffer from growth retardation and immune deficiency. Therefore, shortage of DHA in aquaculture feed results in the degradation of aquacultured fish. Usually, DHA for aquaculture feed is obtained through extraction from tuna eyes or squid skins, and is optionally processed for use as feed additives.

Korean Patent No. 10-338193 discloses Schizochytrium sp. Sπ-2 used to provide DHA as a feed additive. The novel microorganism Schizochytrium, a kind of eukaryote, is provided as a feed for rotifers which are then raised and used to supply DHA for aquaculture fry. U.S. Pat. No. 6,566,123 discloses a process for culturing microorganisms at a low chloride concentration for the production of unsaturated lipids.

### DISCLOSURE OF INVENTION

### Technical Problem

It is an object of the present invention to provide the novel microbes, Thraustochytrium sp. KJS-1, which produce a high content of DHA, and Bacillus polyfermenticus KJS-2, which has highly inhibitory activity against a wide spectrum of bacteria.

It is another object of the present invention to provide an aquaculture feed additive for fisheries, which can provide the essential nutrient DHA for fish and shows antibacterial and antifungal activity, thereby preventing mass death of fish due to bacterial or fungal diseases.

### Technical Solution

In accordance with an aspect of the present invention, there is provided a novel microbe Thraustochytrium sp. KJS-1, deposited at the Korean Culture Center of Microorganisms (KCCM) with Accession No. KCCM 10667P, which produces docosahexanoic acid (DHA).

In accordance with another aspect of the present invention, there is provided a novel microbe Bacillus polyfermenticus KJS-2, deposited at the Korean Culture Center of Microorganisms (KCCM) with Accession No. KCCM 10769P, which has antibacterial and antifungal activity.

In accordance with a further aspect of the present invention, there is provided an aquaculture feed additive for fisheries, comprising a mixture of freeze dried Thraustochytrium sp. KJS-1, freeze dried Bacillus polyfermenticus KJS-2, and freeze dried Bacillus licheniformis in a predetermined weight ratio.

In a preferable modification, the mixture consists of a weight ratio of 5:1:2.6 freeze dried Thraustochytrium sp. KJS-1: freeze dried Bacillus polyfermenticus KJS-2: freeze dried Bacillus licheniformis.

In another preferable modification, the freeze dried Bacillus polyfermenticus KJS-2, and the freeze dried Bacillus licheniformis each contain 1×10¹⁰ colonies per gram and the freeze dried Thraustochytrium sp. KJS-1 contains 77 mg of docosahexanoic acid (DHA) per gram.

### Advantageous Effects

The aquaculture feed additive for fisheries in accordance with the present invention, comprising a mixture of the novel microbes Thraustochytrium sp. KJS-1) and Bacillus polyfermenticus KJS-2 of the present invention with Bacillus licheniformis, not only provides the essential nutrient for fish, but also shows antibacterial and antifungal activity, thereby preventing cultured fish from dying of diseases.

### BRIEF DESCRIPTION OF THE DRAWINGS

FIG. 1 is a phylogenetic diagram of the novel marine microalga Thraustochytrium sp. KJS-1.

FIG. 2 shows a base sequence of 16S rRNA of the novel microbe Bacillus polyfermenticus KJS-2, along with those of 16S rRNA of the same three species (AY9473, DQ597146, and BS).

FIG. 3 is a phylogenetic diagram of the novel Bacillus polyfermenticus KJS-2.

FIG. 4 is a growth curve for Thraustochytrium sp. KJS-1.

FIG. 5 is a growth curve for Bacillus polyfermenticus KJS-2.

FIG. 6 is a growth curve for Bacillus licheniformis.

FIG. 7 shows graphs in which growth yields of Thraustochytrium sp. KJS-1 are plotted against temperatures and salt concentrations.

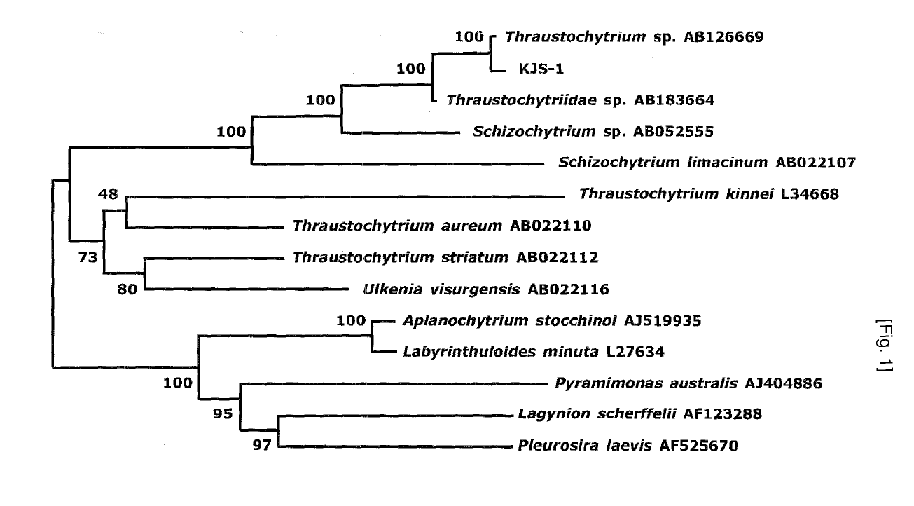

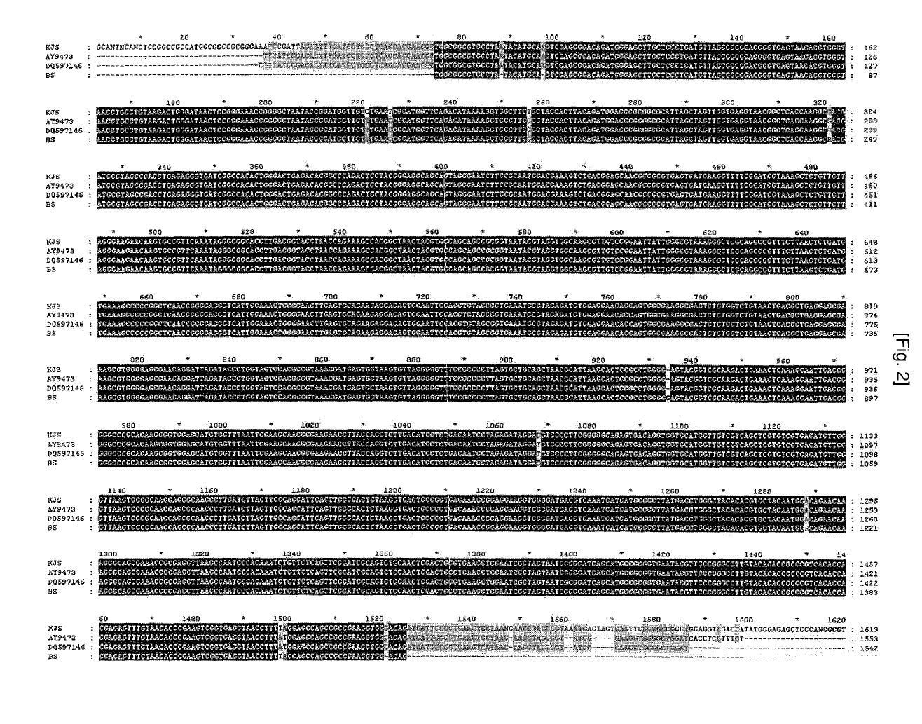

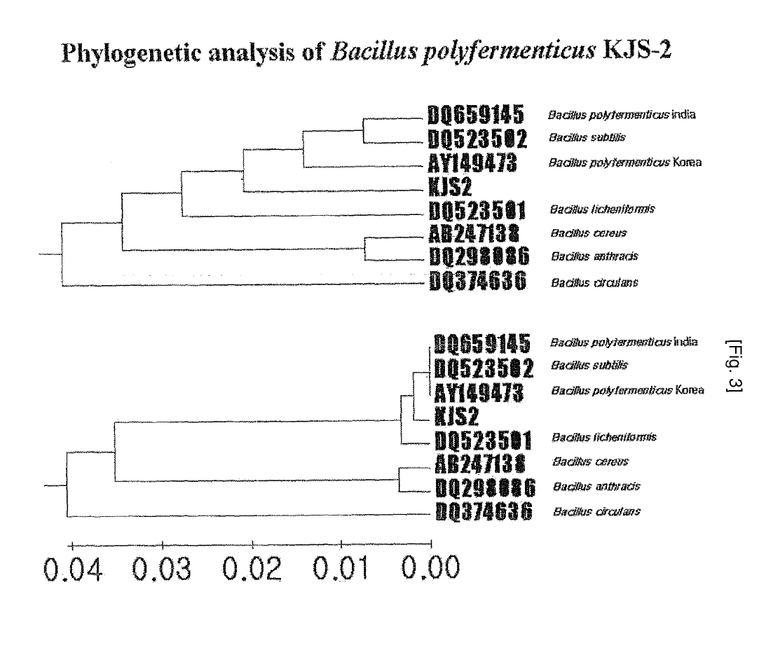

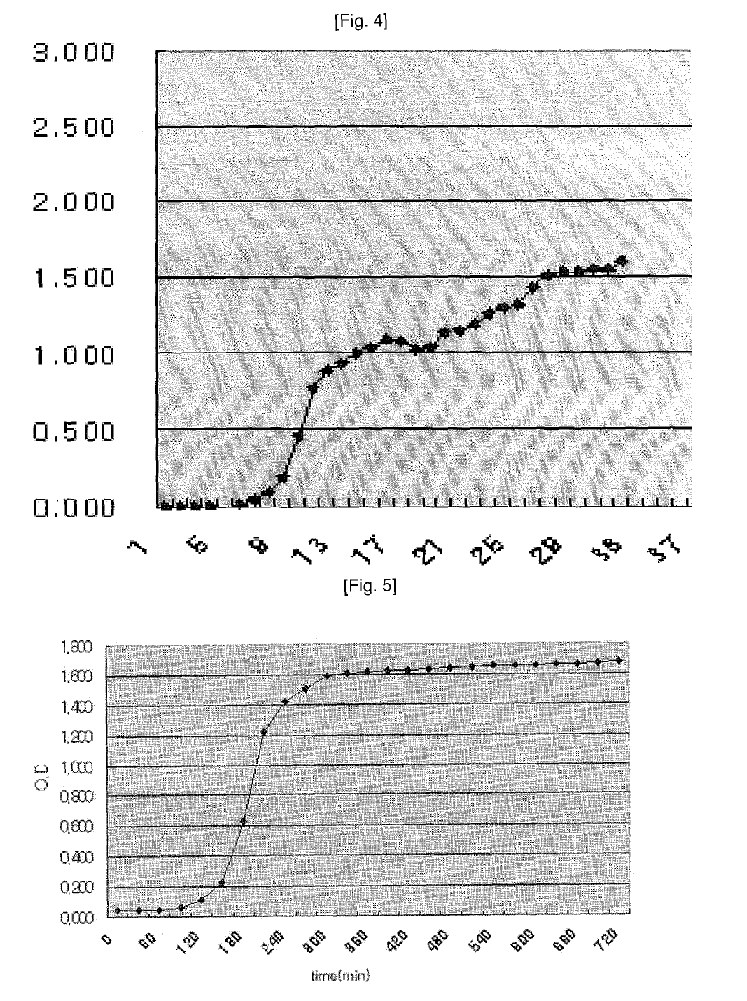

### BEST MODE FOR CARRYING OUT THE INVENTION

Intensive research, conducted by the present inventors, resulted in the discovery of novel microorganisms in the sea south of Korea, which can produce unsaturated fatty acids. Of them, a microbe which produces unsaturated fatty acids at a high yield was selected. Its taxonomy was determined through genetic analysis. 18S rRNA analysis indicated that the novel microorganism belongs to Thraustochytrium sp., a microalga. This novel microalga was named Thraustochytrium sp. KJS-1 (hereinafter referred to as ‘T-1’) and deposited at the Korean Culture Center of Microorganisms (KCCM) on Aug. 10, 2005 with Accession No. KCCM 10667P.

The intracellular DHA which the microalga T-1 produces may be extracted and quantitatively analyzed as follows. First, a solid specimen is dried at 105° C. for 3 hrs, before the addition of 10 ml of an extraction solvent to 1 g of the specimen. A mixture of 2:1 chloroform: methanol is used as the extraction solvent. The resulting solution is concentrated in a vacuum, and the concentrate is esterified with 5 ml of 3.4% hydrochloric acid and methanol. Extraction with 20 ml of hexane is followed by concentration in a vacuum. A solution of the concentrate in 2 ml of ethanol is used as a specimen for gas chromatography. As a result, the intracellular DHA content of the microalga T-1 was found to amount to 7.7%, on average, of the dry weight thereof. For this analysis, the microalga was cultured at 25° C. for 72 hrs in a medium containing 1 liter of sea water, 1 g of yeast extract, 0.5 g of polypeptone, and 50 g of glucose, pH 6-6.5 with stirring at 45 rpm. A distinctive nucleus, characteristic of eukaryotes, was observed with crystal violet staining. The microalga T-1 is difficult to discern from general bacteria as it changes in size and morphology with nutrition. However, when it is stained, the microalga T-1 is clearly different from bacteria under a microscope. Freeze-dried T-1 gives off an odor of anchovies, which is believed to be attractive to fish.

The use of Bacillus polyfermenticus (hereinafter referred to as ‘BP-1’) cultured in broth at 37° C. as a feed additive, is disclosed in Korean Pat. No. 10-458487, granted to the present inventor. According to the patent, the lactobacillus is fermented and fed in the form of spores to animals.

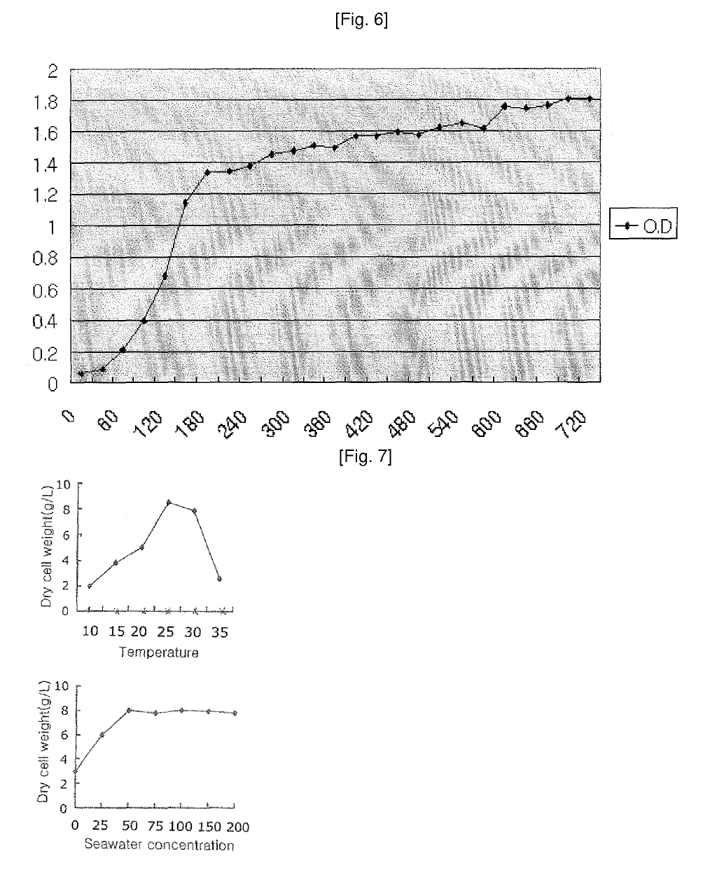

Also, the present inventor found a novel Bacillus polyfermenticus sp which is highly active against bacteria and can live in symbiosis with the other two microbes which are used in the present invention. This novel microbe was named Bacillus polyfermenticus KJS-2 (hereinafter referred to as ‘BP-2’) and was deposited at the Korean Culture Center of Microorganisms (KCCM) on Aug. 16, 2005 with Accession No. KCCM 10769P. BP-1 and BP-2 were observed to differ from each other in colony shape. While BP-1 colonies have a characteristic spread appearance and are pale yellow in color, BP-2 colonies are clear, circular in shape, and slightly darker in color than BP-1. BP-2, with a generation time of 36.9 min, grows faster than BP-1, which shows a generation time of 47 min. Bacillus licheniformis (hereinafter referred to as “BL”) has a generation time of 32 min, shorter than that of BP-2 (see, FIGS. 5 and 6). BP-2 and BP-1 have similar spectra of antibacterial activity. However, BP-2 shows inhibitory activity against Lactococcus garvieae, but BP-1 does not.

BL lives in symbiosis with the two novel bacterial cell lines according to the present invention, showing antibacterial activity particularly against marine pathogens the growth of which BP-2 cannot inhibit. Thus, the use of BL and BP-2 in combination extends the range of antibacterial activity, thus conferring great commercial value. BL is readily found in edible fermented soybeans and is widely used as a host to produce protease. In Korea, this lactobacillus is commercially circulated in the brand name of “Jung Jang Sang” manufactured by A Ju Pharmaceuticals, Korea, as a medication for making the intestine healthy. According to many research reports in Korea, fermented soybeans prepared with BL have the healthful activity of bringing down the blood pressure.

BP-2 and BL can be cultured according to a general culture method. A culture medium for these bacteria contains 3% glucose, 1% water-soluble glucose, 3% peptone, and mineral M9. Mineral M9 consists of 0.002% iron sulfate, 0.002% zinc sulfate, 0.001% copper sulfate, 0.002% manganese sulfate, 0.03% magnesium sulfate, 0.2% ammonium sulfate, 0.1% calcium chloride, 3% dipotassium phosphate, and 3% monopotassium phosphate. In this medium, BP-2 and BL are cultured at 37° C. for 65 hours with stirring at 200 rpm. Freeze drying is conducted to stably store them.

In this invention, BP-2 and BL, which can live symbiotically and broaden the spectrum of antibacterial activity, are used in combination with the marine microalga T-1, which is rich in DHA, in an aquaculture-feed additive for fisheries.

A better understanding of the present invention may be obtained in light of the following examples, which are set forth to illustrate, but are not to be construed to limit the present invention.

In the following examples, characteristics of each cell line and test data on symbiotic relationship and antibacterial activities of BP-2 and BL will be provided. Field tests were performed at Sebo Fisheries, Tongyoung, Korea.

### MODE FOR THE INVENTION

### Example 1

T-1 was cultured for 72 hrs in a medium containing 1 g of yeast extract, 0.5 g of polypeptone and 50 g of glucose per liter of sea water, pH 6-6.5 with stirring at 45 rpm. Culturing was performed at various temperatures to determine the optimal temperature. When incubated at 25° C., T-1 was optimally grown, as its freeze-dried mass amounted to 8 g per liter. Salt concentration is an important factor to determine the growth of T-1 because it is a marine microbe. Although seawater has a salt concentration of 3% (seawater concentration 100), T-1 grew equally well throughout a salt concentration range from 1.5% (seawater concentration 50) to 6% (seawater concentration 200), yielding about 8 g of freeze dried mass per liter. The freeze dried T-1 was pale yellow in color, giving off a characteristic odor similar to that of anchovies. In order to determine the DNA content of T-1, primary extraction of the freeze-dried mass in an organic solvent was followed by esterification with methanol. Secondary extraction with hexane was performed before vacuum concentration. The concentrate was dissolved in ethanol and used for gas chromatography analysis. The DHA content of the freeze-dried T-1 was found to be 7.7% on average.

### Example 2

BP-2 and BL were each cultured in a typical medium containing 3% glucose, 1% water-soluble starch, 3% peptone, and mineral M9. The mineral M9 contained 0.002% ferrous sulfate, 0.002% zinc sulfate, 0.001% copper sulfate, 0.002% manganese sulfate, 0.03% magnesium sulfate, 0.03% ammonium sulfate, 0.1% potassium chloride, 3% potassium dihydrogen phosphate, and 3% potassium monohydrogen phosphate. They were cultured at 37° C. for 65 hrs with stirring at 200 rpm and then freeze dried. The mass of each of the freeze dried bacteria amounted to 2.2-2.31 g.

### Example 3

The novel microbes BP-2 and BL were assayed for antibacterial activity. After being immersed in a trypticase soy broth in which BP-2 or BL had been cultured, a sterile loop of wire was streaked in one line across trypticase soy agar (TSA), which was then incubated for 16 hrs. 10 ml of a TSA medium containing 500 ▭ of marine harmful microbes was poured onto the agar and solidified at room temperature, followed by incubation overnight. Antibacterial activity of BP-2 and BL could be observed with the naked eye as the metabolites produced by BP-2 or BL inhibited the growth of the harmful microbes. Detailed results are as follows.

Both BP-2 and BL showed highly inhibitory activity against Flexibacter tractuosus, Lactococcus garvieae, Vibrio ordalii, and Vibrio vulnificus. Against Streptococcus iniae, BP-2 was found to have low inhibitory activity, while BL showed high antibacterial activity. With regard to Vibrio harveyi, BP-2 was inhibitory but BL was not. In contrast, the growth of Streptococcus parauberis was inhibited by BL but not by BP-2. Therefore, BP-2 and BL can act in a complementary way with each other in conferring antibacterial activity against a spectrum of microbes. In addition, a mixture of 1:1 BP-2:BL was tested in the same manner as described above, and was found to have antibacterial activity against all of the harmful marine microbes tested.

### Example 4

In this example, the feed additive was field tested for fish pathogen treatment and attractive action. 0.75 g of T-1, 0.15 g of BP-2, and 0.39 g of BL (5:1:2.6, weight ratio), all freeze dried, were mixed, and the mixture was added to 72 g of feedstuff for fry (corresponding to about 1.8% of the total weight of the feedstuff). The feedstuff was fed in uniform aliquots three times a day for six days to 200 young fish. Each of the freeze-dried BP-2 and BL contained 1×10¹⁰ colonies per gram. T-1 contained about 77 mg of DHA per gram (refer to Example 1). 200 fish (Acanthopagrus schlegeli), which had been infected with pathogens while reared in marine fish culture cages, were transferred to a fish seeding field (FRP bath). On Day 1, when the feedstuff containing the feed additive according to the present invention was fed in the above-mentioned manner, 8 fish died. 5 and 2 fish died on Day 2 and Day 3. Thereafter, however, no dead fish were found. To scale up the experiment, the feedstuff was fed to 20,000 fish in a 29-ton field. Similar results were obtained, and no dead fish were found after the feedstuff was fed for 3 days. In addition, attractive action was observed when the feedstuff containing the feed additive of the present invention was used, but not when the feedstuff containing only BP-2 and BL was used, leading to the conclusion that the characteristic odor of T-1 attracts fish.

### Example 5

The aquaculture feed additive according to the present invention was assayed for antifungal activity in a filefish hatchery in which red fungi was found. As a control, a commercially available product, such as that manufactured by INVE Aquaticus Health and marketed under the brand name of “Pro-W” was used. It was tested according to the protocol provided by the manufacturer and the amount thereof was 200 ppm. After providing the control for 3 days, no fungi were detected. The aquaculture feed additive of the present invention was used in the same manner as in Example 2, and after the additive was fed for 1.5 days, no fungi were observed. These data, along with the results from the test on the fungus Aspergillus oryzae (see Example 3), demonstrate that the aquaculture feed additive according to the present invention has antifungal activity.

### INDUSTRIAL APPLICABILITY

As described hitherto, the aquaculture feed additive for fisheries in accordance with the present invention, comprising a mixture of the novel microbes Thraustochytrium sp. KJS-1) and Bacillus polyfermenticus KJS-2 of the present invention and Bacillus licheniformis not only provides essential nutrients for fish, but also shows antibacterial and antifungal activity, thereby finding wide applicability in the fishery industry.

1. A novel microbe Thraustochytrium sp. KJS-1, deposited at the Korean Culture Center of Microorganisms (KCCM) with Accession No. KCCM 10667P, which produces docosahexanoic acid (DHA).

2. A novel microbe Bacillus polyfermenticus KJS-2, deposited at the Korean Culture Center of Microorganisms (KCCM) with Accession No. KCCM 10769P, which has antibacterial and antifungal activity.

3. An aquaculture feed additive for fisheries, comprising a mixture of freeze dried Thraustochytrium sp. KJS-1, freeze dried Bacillus polyfermenticus KJS-2, and freeze dried Bacillus licheniformis in a predetermined weight ratio.

4. The aquaculture feed additive according to claim 3, wherein the mixture consists of a weight ratio of 5:1:2.6 freeze dried Thraustochytrium sp. KJS-1: freeze dried Bacillus polyfermenticus KJS-2: freeze dried Bacillus licheniformis.

5. The aquaculture feed additive according to claim 3, wherein the freeze dried Bacillus polyfermenticus KJS-2 and the freeze dried Bacillus licheniformis each contain 1×10¹⁰ colonies per gram and the freeze dried Thraustochytrium sp. KJS-1 contains 77 mg of docosahexanoic acid (DHA) per gram.
